(19) United States
(12) Patent Application Publication
(10) Pub. No.: US 2018/0031569 A1
(43) Pub. Date: Feb. 1, 2018

(54) DEVICES FOR DETECTION OF ANTIBODIES AGAINST THERAPEUTIC DRUGS

(71) Applicant: Maria D. F. S. Barbosa, Philadelphia, PA (US)

(72) Inventor: Maria D. F. S. Barbosa, Philadelphia, PA (US)

(21) Appl. No.: 15/663,650

(22) Filed: Jul. 28, 2017

Related U.S. Application Data
(63) Continuation-in-part of application No. 15/376,775, filed on Dec. 13, 2016.
Application No. 15/376,775 is a continuation-in-part of application No. 15/091,483, filed on Apr. 5, 2016.

(51) Int. Cl. G01N 33/68; G01N 33/74; G01N 33/66

(52) U.S. Cl. CPC G01N 33/6854; G01N 33/66; G01N 33/74; G01N 2333/62; G01N 2800/042

(57) ABSTRACT

Devices for detection of antibodies against therapeutic drugs (ADAs), in which chimeric proteins facilitate immobilization and exposure of target epitopes, are described. Results can be obtained in minutes, enabling point of care testing and/or patient self-testing, and risk assessment. The data can also be used for deriving scientific knowledge in the fields of oncology, autoimmune diseases (including but not restricted to diabetes, multiple sclerosis and rheumatoid arthritis), cardiovascular diseases, rare diseases, and other diseases for which treatment comprises administration of a therapeutic drug and/or gene therapy.

### CLAIM OF PRIORITY

This is a divisional application and claims priority to U.S. application Ser. No. 15/376,775, filed on Dec. 13, 2016, which claims priority to U.S. patent application Ser. No. 15/091,483, filed on Apr. 5, 2016, which claims priority to U.S. Provisional Patent Application No. 62/146,232 filed on Apr. 10, 2015. The respective contents of these patent documents are hereby incorporated by reference in their entirety.

### FIELD OF THE INVENTION

The present invention relates to devices for detection of antibodies against therapeutic drugs (ADAs), and their applications. More specifically, the present invention relates to devices that can be used for point-of-care testing, alone or in combination with monitoring of other analytes.

### BACKGROUND OF THE INVENTION

A competent host immune system is capable of mounting antibody responses against therapeutic agents (“drugs”) perceived as foreign, resulting in the formation of anti-drug antibodies (“ADAs”). Those antibody responses may be intentionally induced (as in the case when the drugs are vaccines against pathogens such as bacteria or viruses), or may be an unintended and unanticipated effect of a therapeutic drug administration (Barbosa, M. D. F. S. and Smith, D. D. 2014 Drug Discov. Today. 19: 1897-1912; expressly incorporated by reference herein).

Surprisingly, in the case of many therapeutic drugs which have already been approved for marketing by regulatory authorities and are considered safe by healthcare professionals and patients, often ADA responses to them may result in adverse reactions of varying negative consequences, for example life-threatening IgE- or IgG-mediated anaphylaxis or anaphylactic shock (Barbosa, M. D. F. S. and Smith, D. D. 2014 Drug Discov. Today. 19: 1897-1912; expressly incorporated by reference herein). They can also decrease or abolish drug efficacy (Barbosa, M. D. F. S. and Smith, D. D. 2014 Drug Discov. Today. 19: 1897-1912; expressly incorporated by reference herein).

Another possible dreadful consequence of ADAs is the neutralization of an endogenous essential protein by cross-reactive ADAs against an administered protein counterpart. A non-limiting example is erythropoietin; neutralizing antibodies to therapeutic erythropoietin have caused red cell aplasia by also neutralizing the endogenous protein counterpart (Woodcock, J. et al. 2007 Nature Reviews in Drug Discovery 6: 437-442; expressly incorporated by reference herein).

Despite unwanted ADAs being a concern in several therapeutic areas, there is no point of care device marketed for ADA detection, and hence it is very rare for a clinician to test patients regarding their ADA status (Kolata, G. The New York Times, May 15, 2017). ADA assays that have been used during preclinical and clinical studies require multiple steps, qualified laboratories, trained personal, storage of frozen or refrigerated reagents, pipetting and other reagent manipulations that are unpractical and conducive to errors. Other concerns about the available ADA assays include but are not limited to the cumbersome nature of blood sample collection, handling, and shipping, and also the length of time to obtain results after the testing is initiated, which can take up to days.

Human insulin is an essential protein hormone secreted by the beta cells of the pancreatic islets. It is formed from a precursor molecule that is cleaved by proteases. The mature insulin consists of fifty-one amino acids, contained within an A chain and a B chain that are held together by two disulfide bridges. Type 1 diabetes patients are dependent on exogenous insulin injections to sustain life, while type 2 diabetes patients often can control the symptoms with diet, exercise and non-insulin medications such as small molecule drugs (Barbosa, M. D. F. S. and Smith, D. D. 2014 Drug Discov. Today 19: 1897-1912; expressly incorporated by reference herein). ADAs can cause insulin resistance. In addition, development of cross-reactive ADAs against recombinant insulin may convert type 2 diabetics to type 1 diabetics. By “recombinant insulin” as defined herein it is meant exogenous insulin, which is produced outside a patient. Said recombinant insulin can be produced in a number of different ways, non-limiting examples are recombinant insulin produced in bacteria or yeast. It can also have modifications, including but not limited to amino acid mutations in relation to human endogenous insulin. Mutations and/or other modifications to an endogenously produced human protein may cause it to be recognized as foreign by a human patient immune system; and ADA response is then likely to ensue.

Cross-reactive antibodies against recombinant insulin that can neutralize endogenous insulin seemingly have been a non-obvious problem. Often healthcare professionals prescribe insulin to type 2 diabetic patients without knowing their ADA status. ADAs developed against the administered drug may be cross-reactive with the endogenous insulin, and the patients may become dependent on insulin injections to sustain life. Currently there are approximately 30 million type 2 diabetics in the US; among them, approximately 1.25 millions are diabetic type 1.

There is no point of care test for anti-insulin antibodies in the market. Assays used to detect ADAs against insulin are difficult to perform and typically involve the use of radioactive material such as iodine-125 (¹²⁵I) radiolabelled insulin (Hamasaki, H. and Yanai, H. 2014 Diabetes and Metabolism 40: 481-482; expressly incorporated by reference herein). For example, blood is collected from a patient and serum prepared from the blood sample; a serum sample is incubated with ¹²⁵I-insulin, and if ADAs against insulin are present in the sample, a complex can be formed between the radiolabeled insulin and the ADAs. Those complexes are precipitated and separated by centrifugation. Radioactive counting of the pellet is used to estimate the presence of anti-insulin antibodies.

¹²⁵I is a radioactive isotope of iodine, which has half-life around 60 days. Well-trained personnel and authorized laboratories are required for handling of radioactive materials. Designated laboratory space is also required, which needs to been clearly labeled and have warning signal. Protective equipment is required for handling of radioactive materials. Containers of ¹²⁵I, including sample vials of iodinated compounds, should always be opened in a fume hood. Typically, iodinations must be performed under surveillance of an Environmental Health and Safety Office, and thyroid count bioassays must be performed following an iodination.

Described in embodiments of the present invention are chimeric proteins that enable immobilization of target antigens without masking them, enabling development of superior assays and devices to monitor ADAs. Chimeric insulin is a non-limiting example. Those chimeric proteins may also have increased stability, a property that makes them suitable for the construction of point of care devices to be shipped and stored at room temperature.

By “chimeric protein” as used herein is meant a drug or a segment of a drug, wherein said drug or segment contains one or more target antigens and is fused with an entity, wherein said entity is from the group consisting of protein, peptide, lipid, carbohydrate, oligonucleotide, chemical entity or moiety. As a non-limiting example, a variable region of a human IgG1 antibody drug fused with the constant region of a mouse IgG2a antibody is a chimeric protein as defined herein; another non-limiting example of chimeric protein is an insulin molecule fused with a constant region of an antibody, or with a chemical entity.

There is an unmet need for devices to readily detect ADAs and to perform risk assessment for biotherapeutics; none is currently marketed for regular testing at point of care facilities or by patients. The present invention provides portable devices for ADA detection, which address above-described challenges, providing results within minutes. Use of chimeric proteins enables efficient immobilization of therapeutic drugs such as insulin, avoiding protein epitope masking and providing superior results. Use of non-human protein regions or other linkers for construction of said chimeric proteins can in some instances play a role in reducing matrix interference. Non-limiting uses for said devices include: risk assessment; stratification of patients likely to benefit from a given therapy; monitoring of therapy safety; drug comparisons; determination of likelihood of drug efficacy; estimation of the need for tolerance induction; postmarketing surveillance.

### BRIEF SUMMARY OF THE INVENTION

This invention is directed to devices to detect antibody responses against therapeutic drugs. In one aspect, portable devices for ADA testing can be used by patients (self-test), or at a point of care such as a physician's office, or during clinical trials.

In another aspect, stable chimeric proteins are used that facilitate immobilization of target antigens without masking them; chimeric insulin is a non-limiting example. The chimeric proteins may also have increased stability, a property that makes them suitable for the construction of point of care devices to be shipped and stored at room temperature.

In another aspect, the devices for ADA detection include an access code for a database, which is in the device itself, or can be accessed on a separate location.

The present invention provides devices that can be used for various applications, including but not restricted to the following: risk assessment; selection of therapeutic drug for patient treatment; evaluation of the need to change therapeutic drug or to apply tolerance regimens; selection of patients for clinical trials; postmarketing surveillance.

### BRIEF DESCRIPTION OF THE DRAWINGS

FIG. 1. Schematic representation of a chimeric protein, which has the constant region of an IgG1 immunoglobulin fused with human insulin.

FIG. 2. Schematic representation of a chimeric protein, which has the constant region of an IgG1 immunoglobulin fused with a mutant human insulin.

FIG. 3. Schematic representation of a chimeric protein, which has the constant region of an IgG1 immunoglobulin fused with a mutant human insulin.

### DETAILED DESCRIPTION OF THE INVENTION

Common therapeutic drugs (thereafter also referred to as “drug” or “drugs”) can be either natural products, or small molecule drugs, or peptides, or therapeutic proteins (biotherapeutics), or small-molecule-biotherapeutic conjugates (Barbosa, M. D. F. S. et al. 2013 Anal. Biochem. 441: 174-179; Barbosa, M. D. F. S. and Smith, D. D. 2014 Drug Discov. Today 19: 1897-1912; Woodcock, J. et al. 2007 Nat. Rev. Drug Discov. 6: 437-442; all expressly incorporated by reference herein). Combination therapies (in which more than one entity is used) are also common. Other drug classes can also include oligonucleotides, lipids, and other entities. Of note, a drug may contain target antigens from the group consisting of protein, peptide, carbohydrate, oligonucleotide, lipid, chemical entity or moiety. Vaccines are also designated as drugs; in this case the ADA responses are intentionally induced.

In order that the invention may be more completely understood, several definitions are set forth below. Such definitions are meant to encompass grammatical equivalents.

By “immunogenicity” as used herein is meant the ability of a protein or another substance or molecule to elicit a host's immune response.

By “tolerance” as used herein is meant immune tolerance to a protein or another substance or molecule, typically characterized by the lack of immune responses.

By “antibody” as used herein is meant a protein that binds an amino acid sequence or another molecular entity. In mammals such as humans and mice, antibodies contain paired heavy and light polypeptide chains. Each chain contains a variable and a constant region. The variable regions of the light and heavy chains are required for binding the target antigen.

By ‘ADA″ or “anti-drug antibody” as used herein is meant antibody that bind to a protein or other molecular-entity or target antigen, whereas that protein or other molecular entity or antigen can be a therapeutic drug. In that sense, all antibodies are essentially “binding”.

By “antigen” as used herein is meant a substance that induces an immune response.

By “target antigen” as used herein is meant the molecule that is bound specifically by the variable region of a given antibody. A target antigen may be from a protein, peptide, carbohydrate, lipid, oligonucleotide, chemical entity or moiety, or a combination of those.

By “low binding propensity” as described herein is meant a decreased affinity for binding, when compared with conditions and entities in which said affinity would be high. Of note, said propensity is not defined in absolute terms. For example, a monoclonal antibody specifically directed to the constant region (“Fc”) of an IgG1 therapeutic human antibody may have a low binding affinity (“K_{D}”) for said Fc region; however, said antibody may have high binding propensity for the human Fc by comparison with the binding to a mouse IgG2a constant region. The amino acid identity between said constant regions of mouse and human is often less than 50%. This non-limiting example is given for better understanding, without constraining the definition to exclude other entities or combinations thereof.

By “K_{D}” as defined herein is meant a dissociation constant, which is specific type of equilibrium constant, and is sometimes use to provide a quantitative measurement of antibody affinity.

By ‘matrix components’ as described herein is meant entities present in a biological sample; as a non-limiting example, serum samples may contain a plethora of proteins, carbohydrates, and/or other components, and the matrix components of a human serum sample can differ from a non-human serum sample.

By “antibody epitope” as used herein is meant a region of the target antigen that binds to the antibody variable region.

By “immunoglobulin (Ig)” herein is meant a protein consisting of one or more polypeptides substantially encoded by immunoglobulin genes. Immunoglobulins may have a number of structural forms, including but not limited to full-length antibodies, antibody fragments, and individual immunoglobulin domains. Immunoglobulins include but are not limited to antibodies.

By “IgG” as used herein is meant a polypeptide belonging to the class of antibodies that are substantially encoded by a recognized immunoglobulin gamma gene. In humans this class comprises IgG1, IgG2, IgG3, and IgG4. Also included are hybrids of IgG proteins in which amino acids for one IgG protein are substituted for amino acids of a different IgG protein (e.g. IgG1/IgG2 hybrids).

By “amino acid” as used herein is meant one of the 20 naturally occurring amino acids or any non-natural analogues that may be present at a specific, defined position.

By “protein” herein is meant attached amino acids. The protein may be made up of naturally occurring amino acids and peptide bonds, or synthetic peptidomimetic structures, i.e. “analogs”, such as peptoids (see Simon, R. J. et al. 1992 Proc. Natl. Acad. Sci. USA 89: 9367-9371). Thus “amino acid”, or “peptide residue”, as used herein encompasses both naturally occurring and synthetic amino acids.

By “database” as used herein is meant a structured combination of information and/or data analyses, which can be accessed in one or more ways.

By “assay” as used herein is meant a procedure for testing samples.

By “ADA portable device” or “portable device” as used herein is meant a portable device of the present invention, which allow for testing samples regarding the presence of ADA, and that can also have additional features including but not limited to an associated database and/or capabilities to measure other metabolites.

By “composite device” as use herein is meant a device that combines more than one analyte detection method or theory of operation.

By “PEGylation” as used herein is meant the addition of one or more polyethylene glycol (PEG) moiety by various means that may comprise the use of linkers.

Antibodies (also named immunoglobulins) are proteins that bind a specific antigen. In mammals such as humans and mice, antibodies contain paired heavy and light polypeptide chains. Standard antibody structural units typically comprise a tetramer. Each tetramer is usually composed of two identical pairs of polypeptide chains, each pair having one “light” (typically having a molecular weight of about 25 kDa) and one “heavy” chain (typically having a molecular weight of approximately 50 kDa).

By “isotype” as used herein is meant any of the subclasses of immunoglobulins. The known human antibody isotypes are IgG1, IgG2, IgG3, IgG4, IgA1, IgA2, IgM1, IgM2, IgD, and IgE.

Each antibody chain contains a variable and a constant region, as described above. The variable regions of the light and heavy chains are required for binding the target molecule (the antigen). All ADAs are capable of binding to a target molecule, and hence are referred to as binding antibodies.

The following Igs are typically observed in higher mammals: IgD, IgA, IgE, IgM and IgG. IgD amounts to a small percentage of total serum Igs (less than 1%); IgA and IgM can comprise approximately 10-20%. IgG is the predominant Ig in blood. IgM is generally known as the early antibody, as it precedes the IgG response.

By “full length antibody” as used herein is meant the structure that constitutes the natural biological form of an antibody, including variable and constant regions, including one or more modifications. Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each. In certain variations, antibody may mean a protein consisting of one or more polypeptides substantially encoded by all or part of the recognized immunoglobulin genes. Antibody herein is meant to include full-length antibodies and antibody fragments, and may refer to a natural antibody from any organism, an engineered antibody, or an antibody generated by recombinant techniques for experimental, therapeutic, or other purposes.

A “patient” for the purposes of the present invention includes both human and other animals, preferably mammals and most preferably humans.

The term “treatment” in the present invention is meant to include therapeutic treatment, as well as prophylactic, or suppressive measures for a disease or disorder. “Treatment” also encompasses administration of a therapeutic drug after the appearance of the disease in order to ameliorate, control, or to eradicate the disease. Those “in need of treatment” include mammals already having the disease or disorder, as well as those prone to having the disease or disorder, including those in which the disease or disorder is to be prevented.

By “adverse event” as defined herein is meant any undesirable experience (i.e. a bad side effect) associated with the use of a medical product in a patient.

Host antibody responses against an antigen are typically polyclonal, comprising immunoglobulins that bind the antigen with various affinities and/or avidities. Hence, the assays used to detect antibody responses against therapeutic drugs are inherently qualitative, because there is no positive control antibody that would accurately represent all diverse antibodies in each of the samples collected from diverse sources and/or at various times following antigen exposure.

Some embodiments of the present invention include devices to anticipate and detect host immune reactions against therapeutic drugs, and to perform risk assessment for those therapeutic entities. Those devices to detect antibodies against therapeutic drugs can enable self-testing and/or testing at a point of care such as at physician's office, hospital or emergency room. That information regarding the presence or absence of antibodies against the drug can be used independently or combined with, as a non-limiting example, data available in a database. In one embodiment, a code provided with the portable device allows access to a database. Said database can be on the device itself or accessed on a different location.

Detection of Anti-Drug Antibodies (ADAs):

In one embodiment, an application is described that provides a means of utilizing a portable device to detect ADAs for individual testing. Validation of the portable devices may include using them for tests with clinical samples, and comparison with other assays known in the art. In a preferred embodiment, the device is constructed using one or more chimeric proteins. In another embodiment, a non-target antigen region of the chimeric protein is the constant region of an IgG molecule (Capon, D. J. et al. 1989 Nature 337: 525-531; expressly incorporated by reference herein). In other embodiments, a non-target antigen region of the chimeric protein is from a group consisting of protein, peptide, carbohydrate, oligonucleotide, chemical entity or moiety.

The devices of the present invention can detect ADAs by generating a signal, for example when an electrical property is altered upon binding of ADA (U.S. Pat. No. 4,219,335; expressly incorporated by reference herein). Said electrical property includes one or more of the following: resistance; impedance; capacitance; electrical potential. Carbon nanotube biosensors are also included within embodiments of the present invention (Cao, Q. et al. 2017 Carbon nanotube transistors scaled to a 40-nanometer footprint. Science 356: 1369-1372). Binding of ADAs alters the electronic property of the nanotube transistors. A plethora of detectors have also been described which can be adapted to register the signal generated upon binding of ADAs to the target antigen. Paper-based microfluidic technology is also within the scope of the present invention (Dungchai, W. et al. 2009 Analytical Chemistry 81:5821-5826; Parks, T. S. et al. 2017 SLAS Technology 22: 7-12; all expressly incorporated by reference herein).

In another embodiment, the device is used for detection of ADA in blood (U.S. Pat. Nos. 4,594,327; 5,939,331; 5,753,497; all expressly incorporated by reference herein).

In general, the device can have a solid support associated with an analyte-capturing reagent, and said capturing reagent can be incorporated into the device in a number of ways; examples of non-limiting embodiments are provided. In one embodiment the capturing reagent is attached to carbon nanotubes. In another embodiment the capturing reagent is connected to carbon nanotubes by a chemical process. In another embodiment the capturing reagent is dispersed in carbon nanotubes. In another embodiment the capturing reagent is attached to a membrane. The solid support can be a porous material, for example paper or fabric, or other. In another embodiment, electrodes are attached to the solid support. In another embodiment other forms of detection, for example, a chemical reaction, or fluorescence, or visual signal can be used. The solid support can have any structural configuration as long as the capturing reagent can be attached to the solid support, either directly or by means of other materials, and when attached the capturing reagent is capable of binding to an analyte. In a preferred embodiment, the capturing reagent is a chimeric protein.

It is known that some diabetes patients may have anti-insulin antibodies prior to receiving any insulin drug (pre-existing antibodies) (Barbosa and Smith 2014. Drug Discovery Today 19: 1897-1912; expressly incorporated by reference herein). In one embodiment the ADA testing devices of the present invention can have an important role in risk assessment prior to administering insulin to patients.

ADA incidence against chronically administered products such as insulins and enzyme replacement therapies without monitoring ADAs is also of great concern even if there is no immediate adverse reaction. When the drug dosage is increased to compensate for ADAs, the chronic administration may result in immune complexes not being cleared, leading to immune complex disease and/or other syndromes (Barbosa, M. D. F. S. and Smith, D. D. 2014 Drug Discov. Today 19: 1897-1912; expressly incorporated by reference herein). In such cases, knowledge of ADA incidence and monitoring can provide an effective mechanism to evaluate risk and/or the need for tolerance induction regimens.

Although prophylactic vaccines are intended to induce antibody responses, they prolific unnecessary use may also be problematic. In particular, there is no point of care testing to determine antibody levels against infectious diseases, prior to new vaccine shots to preserve immunity, or during the course of repeated vaccinations. This may lead to unnecessary vaccinations. It should be noted that in some instances antibodies may result in immune complexes involved with the etiology of diseases (Barbosa, M. D. F. S. and Smith, D. D. 2014 Drug Discov. Today. 19: 1897-1912; expressly incorporated by reference herein).

ADAs against adalimumab (Humira®), infliximab (Remicade®) and etanercept (Embrel®) are non-limiting examples of ADAs that can decrease or abolish the efficacy of therapeutic drugs. Adalimumab and infliximab are IgG₁ antibodies that differ regarding their variable region, although they both are tumor necrosis factor (TFN) blockers. Etanercept is a fusion protein of human TNF receptor-2 and human IgG1 Fc (Barbosa, M. D. F. S. and Smith, D. D. 2014 Drug Discov. Today 19: 1897-1912; expressly incorporated by reference herein).

Hosts such as humans and test animals can also mount ADA responses against entities added to therapeutic proteins. For example, anti-polyethylene glycol (anti-PEG) ADAs have been often observed when hosts are dosed with therapeutic drug-PEG conjugates (Barbosa, M. D. F. S. et al. 2013 Anal. Biochem. 441: 174-179; expressly incorporated by reference herein).

In attempts to improve efficacy and/or to protect intellectual property positions, several new versions of marketed therapeutic drugs have been developed. In some instances, the novelty consists of introducing mutations to existing protein drugs. For example, new insulins are now available for treatment of diabetes, which contain mutated protein sequences relative to native insulin. Protein mutations may significantly alter the drug properties (including but not restricted to aggregation propensity), and may also create epitopes involved in T cell activation and unwanted anti-drug antibody (ADA) responses. Unwanted immunogenicity is also a concern for biosimilar versions of marketed protein drugs, including but not restricted to insulin (a “biosimilar” is a biotherapeutic similar to another one already marketed for which the patent has expired).

Monitoring of glucose levels is a necessity for diabetes patients, and typically daily glucose monitoring is required for type 1 diabetics. The implications of ADA incidence and monitoring have been discussed above. This invention is also directed to “composite devices”, which allow monitoring of both glucose levels and ADA, or of insulin and ADA, or of all three, namely ADA, insulin and glucose. There are numerous detectors and glucose meters that can be integrated with the ADA device, to provide results for a complete panel. Typically a number is registered following glucose readings. While the nature of the ADA assay is qualitative, it allows for monitoring ADA levels based on the patient baseline ADA readings. The ADA testing device can also be integrated with insulin pumps, which provide continuous monitoring of glucose and regulated dispensing of insulin.

It is known that diabetes patients can have anti-insulin antibodies prior to receiving any insulin treatment (pre-existing antibodies); juvenile new-onset diabetes patients can have anti-insulin antibodies, whose incidence inversely correlates with age, and genetics has been implicated in risk of juvenile diabetes (Barbosa, M. D. F. S. and Smith. D. D. 2014 Drug Discovery Today 19: 1897-1912; Wang, J. et al. 2011 J. Diabetes 3: 132-137; Groop, L. and Pociot, F. 2014 Mol Cell Endocrinol. 382: 726-739; all expressly incorporated by reference herein). In one embodiment, the portable device can be used to monitor anti-insulin antibodies prior to and during the course of treatment, enabling trend analysis, and collectively providing means of identifying genetic associations. In another embodiment, data obtained with the ADA testing devices can be used for population analysis with patients treated with different drug, alone or in combination with genetic analysis. In another embodiment, the data can be used to guide gene therapy.

Databases built within the devices can be used, registering readings and providing continuous monitoring data. In another embodiment the genetic make up of the patient can be registered within the portable device.

Non-limiting examples of modifications to increase sensitivity and accuracy of the portable device include optimization of the detection method and of sample collection and size, minimization of nonspecific background signal, optimization of materials used for device construction, optimization of reagent concentration immobilized on the device, selection of time for assay development and signal reading. In another embodiment, modifications are made to improve biophysical properties of the reagents used for the device construction, comprising one or more of the following: stability, solubility, and oligomeric state. Improvements in device performance are included within embodiments of the present invention.

The ADA-testing devices of the present invention may be compared with one or more conventional assay used for a given drug, such as for example a radioimmunoassay to test for antibodies against insulin or another assay relevant for comparisons (Berson, S. A. and Yalow, R. S. 1957 Diabetes 6: 402-405; Berson, S. A. and Yalow, R. S. 1957 J. Clin. Invest. 36: 642-647; Berson, S. A. and Yalow, R. S. 1958 Am. J. Med. 25: 155-159; Berson, S. A. and Yalow, R. S. 1996 Obes. Res. 4: 583-600; Hamasaki, H. and Yanai, H. 2014 Diabetes Metab. 40: 481-482; all expressly incorporated by reference herein). The parameters tested may include but are not limited to factors such as sensitivity, robustness, inter and intra assay variation, precision, sensitivity, matrix interference, cut point determination, minimal required dilution, and drug inhibition of the assay (Barbosa, M. D. F. S. et al. 2006 Clin. Immunol. 118: 42-50; Barbosa, M. D. F. S. et al. 2012 J. Immunol. Methods 384:152-156; all expressly incorporated by reference herein).

In another embodiment, the ADA-testing devices of the present invention contain information allowing access to the database. That information can be provided in various manners, for example, as a code on the device case, and/or in its interior, on a user manual, and/or on its packaging. In another embodiment, the database is on the device.

The ADA-testing devices of embodiments of the present invention may be further validated in clinical and/or preclinical studies. That validation may include but not be restricted to comparison of data obtained with samples from the same humans or animal models, tested with an ADA-testing device of the present invention and another assay known in the art or newly invented. Other forms of ADA-testing device validation may also be used.

The ADA-testing devices of the present invention can be used alone to provide information of the ADA positive or negative status or can be used in conjunction with a database and with statistical analyses to infer the probability of safety or efficacy issues due to ADA responses. Those uses are included in embodiments of the present invention.

In another embodiment, the ADA testing devices of the present invention can be used to guide selection of therapeutic drug dose.

In another embodiment, the ADA testing device of the present invention and/or corresponding database can be used to select patients for clinical trials, including but not restricted to clinical development of novel therapeutic proteins, biosimilars or biobetters. By “biosimilar” as defined herein it is meant a therapeutic protein similar to another one already marketed for which the patent has expired (the reference product). By “biobetter” as defined herein it is meant a newer and presumably improved version of a marketed therapeutic protein.

By “pre-existing antibody” as used herein, is meant an antibody against a therapeutic drug or other entity that was present in the body of a human or animal prior to exposure to or administration of that therapeutic drug. In another embodiment, the device of the present invention can be used to test pre-existing antibodies in humans or animals. Data collected may be used for statistical analyses to investigate correlations.

Examples are provided below. These examples are not to be construed as limiting.

### Example 1

Portable Devices for Detection of Anti-Insulin Antibodies are Constructed, which Contain Target Antigens within Chimeric Proteins.

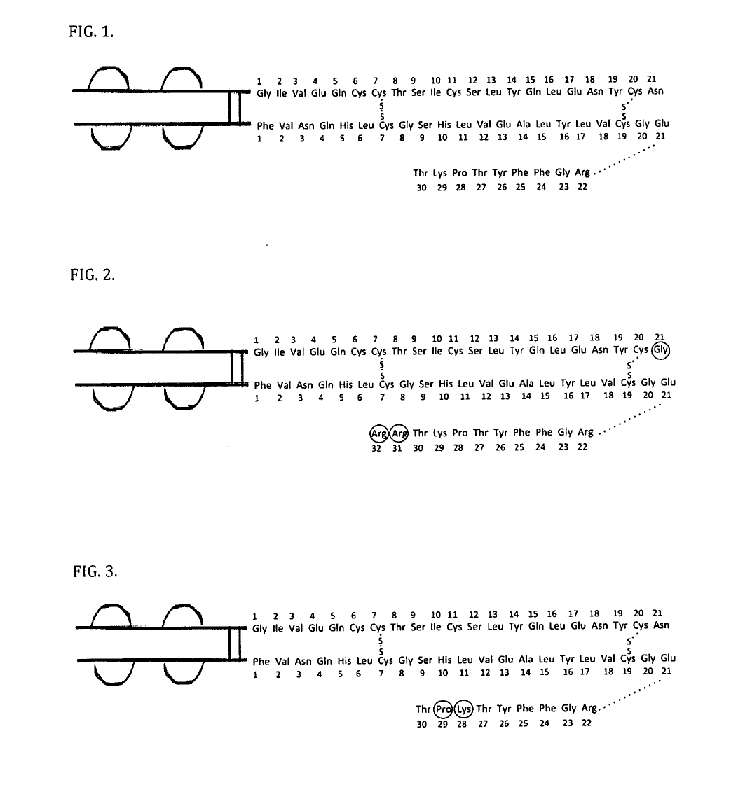

FIG. 1. shows a chimeric protein used for construction of a device of the present invention. In this non-limiting example, the target antigen region consists of human insulin, and it is fused with the constant region of human IgG1. The insulin region is responsible for binding to anti-insulin antibody and generation of a signal, while the non-insulin region allows immobilization on a support material, without masking target antigens.

FIG. 2. shows a chimeric protein used for construction of a device of the present invention. In this non-limiting example, the target antigen region consists of a mutated human insulin, in which the amino acid asparagine at position 21 of the insulin A-chain is replaced by glycine, and two arginines are added to the C-terminus of the B-chain. Said mutated insulin is fused with the constant region of human IgG1. The insulin region is responsible for binding to anti-insulin antibodies and generation of a signal, while the non-insulin region allows immobilization on a support material, without masking target antigens.

FIG. 3 shows a chimeric protein used for construction of a device of the present invention. In this non-limiting example, the target antigen region consists of a human insulin analog, in which the amino acids at positions 28 and 29 on the insulin B-chain are reversed. Said mutated insulin is fused with the constant region of human IgG1. The insulin region is responsible for binding to anti-insulin antibodies and generation of a signal, while the non-insulin region allows immobilization on a support material, without masking target antigens.

1. A device for testing anti-drug antibodies (ADAs) in tissues and body fluids, comprising:
a. A solid support;
b. A material containing one or more immobilized chimeric proteins, wherein the chimeric protein has one or more target antigens; and
c. One or more sensors for a signal generated by binding of target antigens with ADAs.

2. The device of claim 1, wherein the target antigen of the chimeric protein is from the group consisting of protein, peptide, carbohydrate, lipid, oligonucleotide, chemical entity or moiety.

3. The device of claim 1, wherein the chimeric protein has one region with one or more target antigens and a second region with low binding propensity for ADAs.

4. The device of claim 1, wherein the chimeric protein has one region with one or more target antigens and a second region with low binding propensity for components of the sample matrix and for ADAs.

5. The device of claim 1, wherein the target antigen is from the variable region of an antibody.

6. The device of claim 1, wherein the target antigen is from a chemical entity or moiety.

7. The device of claim 1, wherein the target antigen is from a protein molecule.

8. The device of claim 1, wherein the target antigen is from a peptide molecule.

9. The device of claim 1, wherein the target antigen is from an oligonucleotide molecule.

10. The device of claim 1, wherein the target antigen is from a carbohydrate molecule.

11. The device of claim 1, wherein the target antigen is from a lipid molecule.

12. The device of claim 1, wherein the target antigen is from an insulin molecule.

13. The device of claim 1, wherein the target antigen is from an interferon-beta molecule.

14. The device of claim 1 having a code allowing access to a database, wherein said database is on the device itself or is accessed in a separate location.

15. The device of claim 1, wherein said device can detect immunoglobulins from the group consisting of IgG, IgM, IgE, immunoglobulin isotypes.

16. The device of claim 1, connected to a glucose-monitoring device, wherein results for blood glucose levels and ADAs can be obtained using said composite device.

17. The device of claim 1, connected to an insulin-monitoring device, wherein results for blood insulin concentration and ADAs can be obtained using said composite device.

18. The device of claim 1, connected to a glucose-monitoring device and to an insulin-monitoring device, wherein results for glucose concentration, insulin concentration and ADAs in blood can be obtained using said composite device.


SEQUENCE LISTING (5 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 21 aa, Artificial Sequence
  mutated human insulin A-chain
GIVEQCCTSICSLYQLENYCG

SEQ ID NO: 4 - protein, 32 aa, Artificial Sequence
  mutated human insulin B-chain
FVNQHLCGSHLVEALYLVCGERGFFYTPKTRR

SEQ ID NO: 5 - protein, 30 aa, Artificial Sequence
  mutated human insulin B-chain
FVNQHLCGSHLVEALYLVCGERGFFYTKPT